(19) United States
(12) Patent Application Publication
(10) Pub. No.: US 2012/0197117 A1
(43) Pub. Date: Aug. 2, 2012

(54) THERMOACOUSTIC SYSTEM FOR ANALYZING TISSUE

(75) Inventors: Paul A. Picot, London (CA); Michael M. Thornton, London (CA); David A. Steinberg, Milton, MA (US)

(73) Assignee: ENDRA, INC., Ann Arbor, MI (US)

(21) Appl. No.: 13/321,386

(22) PCT Filed: May 19, 2010
(86) PCT No.: PCT/US10/35475

§ 371 Date: Mar. 12, 2012

Related U.S. Application Data
(60) Provisional application No. 61/179,467, filed on May 19, 2009.

(51) Int. Cl. A61B 8/00

(52) U.S. Cl. 600/438

(57) ABSTRACT

Methods and systems to analyze soft tissue or vasculature in a subject, form images with enhanced soft tissue contrast, determine blood flow parameters in soft tissue or vasculature, and to aid in the diagnosis of disease using thermoacoustic methods. Pulsed electromagnetic energy is administered to tissue to excite a thermoacoustic signal in the soft tissue or vasculature. An acoustic receiver or receiver array is coupled to a subject to detect and record the thermoacoustic signals produced. Thermoacoustic data are acquired after administration of a physiologically-tolerable tracer or contrast agent. The acquired data may be analyzed to produce images of the soft tissue and vasculature (angiogram), to determine blood flow parameters, and/or to diagnose disease in a subject.

### CROSS-REFERENCE TO RELATED APPLICATION

This application claims the benefit of U.S. Provisional Patent Application No. 61/179,467, filed on May 19, 2009, herein incorporated by reference.

### BACKGROUND OF THE INVENTION

In general, the system and methods described herein relate to the analysis and visualization of soft tissue and vasculature in a subject, the calculation of blood flow parameters in tissue, and the diagnosis, assessment, and monitoring of disease using thermoacoustic methods.

Blood vessel morphology and tissue perfusion can indicate states of health in organs and can be used for diagnosis of disease and monitoring of treatment. Measurement of blood flow in tissue can be used to diagnose several disorders or disease states including renal disease, cardiovascular disease, stroke, and cancer.

Thermoacoustic imaging uses short pulses of electromagnetic energy to heat absorbing features within an object rapidly, which in turn induces an acoustic pressure wave that can be detected using acoustic receivers. These acoustic waves are analyzed through signal processing, and further processed for presentation and interpretation by an operator.

The perfusion of blood in tissue is a key parameter in characterizing the type, state, or health of the tissue. The differential filling of tissues with an exogenous imaging agent is commonly used in clinical practice to identify tissue abnormalities across multiple imaging modalities (nuclear imaging, magnetic resonance, X-ray, computed tomography, ultrasound, and PET). Typically, the exogenous imaging agent is administered by venous injection. The imaging agent may remain in the blood pool or in some cases may migrate through the vessel wall into the interstitial space. Tracer kinetics methods are established and are an accepted method to estimate perfusion.

In one common method of determining the perfusion of blood, the flow of blood in tissue is measured using tracer kinetics methods. An exemplary method uses a sequence of X-ray computed tomography images to measure the progression of an iodinated contrast agent that was injected into the vasculature. Briefly, a system measures the flow of a quantity of tracer (contrast agent) through tissue. With knowledge of the amount of injected tracer and a measure of the input waveform, estimates of blood flow, blood volume, and mean transit time can be made. The permeability-surface area product of the tissue can also be estimated. Together, these measurements characterize the blood flow properties of tissue, which can be used to classify tissue, and can be used for diagnostic purposes. This method has several drawbacks, including patient exposure to ionizing radiation, contrast agents that may not be physiologically tolerable, high operating costs of equipment, and large equipment requiring a specialized facility. Magnetic resonance imaging can also be used to derive perfusion measurements. This method suffers from many of the same drawbacks as X-ray computed tomography perfusion measurement. Thus, there is a need for new imaging methods.

The thermoacoustic methods of the invention have several advantages over previous methods. The thermoacoustic methods described herein modify the endogenous tissue contrast and may be used to analyze soft tissue and/or vasculature, estimate blood flow and perfusion, and produce increased-contrast angiographic images and images of various soft tissues in the body. The provided thermoacoustic methods may also be used to diagnose disease in a subject (e.g., cardiovascular disease, kidney disease, stroke, and cancer).

### SUMMARY OF THE INVENTION

In a first aspect, the invention provides methods for analyzing soft tissue or vasculature of a subject requiring the steps of: coupling an ultrasound transducer to the subject; delivering to the subject a contrast agent that changes a thermoacoustic signal generated by the soft tissue or vasculature; irradiating the soft tissue or vasculature with electromagnetic energy to generate the thermoacoustic signal; and detecting the thermoacoustic signal, thereby analyzing the soft tissue or vasculature using the thermoacoustic signal. In specific embodiments of the invention, the electromagnetic energy excludes UV light (10 nM to 400 nM) or energy wavelengths of less than 10 nM. In various examples of the above methods, a region of interest in the subject is irradiated.

In additional embodiments of the above methods, the tissue irradiated is selected, without limitation, from heart, kidney, lung, esophagus, thymus, breast, prostate, brain, muscle, nervous tissue, epithelial tissue, bladder, gallbladder, intestine, liver, pancreas, spleen, stomach, testes, ovaries, and uterus. In various embodiments of the above methods, the delivery of the contrast agent occurs by bolus injection or by manual or mechanical infusion.

A single pulse or multiple pulses of electromagnetic energy may be employed in the methods. Individual pulses may have a width between 1 nanosecond and 10 microseconds, e.g., 1 microsecond. Multiple pulses in a series may or may not have the same pulse width. The interval between pulses may or may not be uniform.

Additional embodiments of all the above methods further include generating a two- or three-dimensional image from the detected thermoacoustic signal. For example, the methods may include generating a series of two- or three-dimensional images over time. The time interval between said two or more images may be uniform (constant time interval) or non-uniform (varying time interval). Such methods may be used to create a cineloop, as is known in the art.

Additional embodiments of the above methods may include determining one or more blood flow paramaters from the vasculature (e.g., one or more parameters selected from the group of Blood Flow (BF), Mean Transit Time (MTT), and/or Tissue Permeability-Surface Area product (PS)). In an additional embodiment, the determining of the one or more blood flow parameters includes the step of generating a two- or three-dimensional image of the vasculature (e.g., an image that shows the location and size of the blood vessels). Additional examples of these methods further include generating a series of two or more images of the vasculature over time (e.g., using a uniform time interval or a non-uniform time interval).

In additional embodiments of the above methods, the analysis is indicative of a disease in the subject (e.g., cardiovascular disease, kidney disease, liver disease, stroke, or cancer). In additional embodiments of these methods, the cancer may be selected from the group of hepatocellular carcinomas, metastases, intrahepatic cholangiocarcinomas, liver hemangiomas, nonhemangiomatous benign lesions, adrenocortical carcinoma, anal cancer, appendix cancer, astrocytoma, atypical teratoid/rhabdoid tumor, basal cell carcinoma, bile duct cancer, bladder cancer, bone cancer, brain stem glioma, brain tumor, breast cancer, bronchial tumor, Burkitt lymphoma, carcinoid tumor, cervical cancer, chordoma, chronic lymphocytic leukemia, chronic myeloproliferative disorder, colon cancer, colorectal cancer, craniopharyngioma, cutaneous T-cell lymphoma, endometrial cancer, ependymoblastoma, ependymoma, esophageal cancer, Ewing sarcoma, extracranial germ cell tumor, extragonadal germ cell tumor, extrahepatic bile duct cancer, eye cancer, gallbladder cancer, gastric cancer, gastrointestinal cancer, germ cell tumor, gestational trophoblastic tumor, glioma, hairy cell leukemia, head and neck cancer, hepatocellular cancer, histiocytosis, Hodgkin lymphoma, hypopharyngeal cancer, intraocular melanoma, islet cell tumor, Kaposi sarcoma, kidney cancer, Langerhans cell histiocytosis, laryngeal cancer, acute lymphoblatic leukemia, chronic lymphocytic leukemia, lip and oral cavity cancer, liver cancer, lung cancer, non-Hodgkin lymphoma, macroglobulinemia, osteosarcoma, medulloblastoma, melanoma, merkel cell carcinoma, mesothelioma, mouth cancer, mycosis fungiodes, myelodysplastic syndrome, multiple myeloma, nasal cavity and paranasal sinus cancer, nasopharyngeal cancer, non-small cell lung cancer, oral cancer, oropharyngeal cancer, osteosarcoma, ovarian cancer, ovarian epithelial cancer, pancreatic cancer, papillomatosis, parathyroid cancer, penile cancer, pharyngeal cancer, pituitary tumor, prostate cancer, rectal cancer, renal cell cancer, retinoblastoma, rhabdomycosarcoma, salivary gland cancer, sarcoma, skin cancer, small intestine cancer, soft tissue sarcoma, testicular cancer, throat cancer, thomoma, thymic carcinoma, thyroid cancer, urethral cancer, uterine cancer, vaginal cancer, and Wilms tumor.

In any of the above methods, the electromagnetic energy may be pulsed radiofrequency (e.g., 26 MHz to 1000 MHz) or microwave (e.g., 1 GHz to 10 GHz) electromagnetic energy.

In additional embodiments of the above methods, the contrast agent has increased absorption of electromagnetic energy (e.g., radiofrequency, visible light, near infra red light, or microwave) compared to the absorption of the tissue or vasculature. For example, the contrast agent with increased absorption of microwave or radiofrequency electromagnetic energy has an increased dielectric absorption or an increased ionic conductivity compared to the endogenous tissue, or vasculature, e.g., blood (e.g., a hypertonic solution, such as 5× physiological saline). Additional examples of contrast agents that have increased absorption of radiofrequency electromagnetic energy are agents that contain a ferromagnetic or ferrimagnetic molecule (e.g., ferric ammonium citrate, ferric chloride, ferric citrate, ferric phosphate, ferric pyrophosphate, ferric sulfate, ferrous ascorbate, ferrous carbonate, ferrous citrate, ferrous fumurate, ferrous gluconate, ferrous sulfate, and elemental iron). In various embodiments of the invention, the use of iron oxide particles is excluded. Other specific examples of contrast agents are provided herein.

In alternative embodiments of the above methods, the contrast agent has decreased absorption of electromagnetic energy (e.g., radiofrequency, visible light, near infrared light, or microwave) compared to the absorption of the tissue or vasculature. For example, the contrast agent with decreased absorption of microwave or radiofrequency electromagnetic energy has a decreased dielectric absorption or a decreased ionic conductivity compared to the tissue or vasculature, e.g., blood (e.g., a hypo-ionic solution, such as a hypo-ionic solution that is also an isotonic solution). Non-limiting examples of hypo-ionic solutions include de-ionized osmolarity-balanced water, a solution containing safflower oil, or an aqueous solution containing mannitol, dextrose, or glycerol. In all the above examples, the terms hyperionic and hypo-ionic solutions are relative to the physiological state (e.g., relative to physical properties of blood or tissue).

The invention further provides systems to analyze soft tissue or vasculature in a subject containing: an injector for delivering a contrast agent to the subject; an ultrasound receiving transducer or transducer array; an electromagnetic energy transmitter or transmitter array, wherein the electromagnetic energy transmitter or transmitter array administers pulsed electromagnetic energy to excite a thermoacoustic effect in the soft tissue or vasculature; and hardware or a computer containing software to process the thermoacoustic signal generated by the soft tissue or vasculature. In one embodiment, the electromagnetic energy is pulsed radiofrequency (e.g., 26 MHz to 1000 MHz) or microwave (e.g., 1 GHz to 10 GHz) energy.

Additional embodiments of the systems further contain hardware or a computer containing software for generating a two- or three-dimensional image from the thermoacoustic signal. For example, the hardware or computer may generate a series of two or more images over time. In certain embodiments, the time interval between the two or more images may be uniform or non-uniform.

An additional embodiment of the system further comprises hardware or a computer containing software that synchronizes the delivery of the contrast agent by the injector and the acquisition of the thermoacoustic signal by the ultrasound receiving transducer or transducer array.

Another embodiment of the system further comprises hardware or a computer containing software for determining one or more blood flow parameters including Blood Flow (BF), Blood Volume (BV), Mean Transit Time (MTT), and/or Tissue Permeability-Surface Area (PS) from thermoacoustic signals received by said ultrasound receiving transducer or transducer array.

In additional embodiments of the system, the electromagnetic energy transmitter or transmitter array is pre-formed for a specific body part. In another embodiment of the system, the ultrasound receiving transducer or transducer array is located in an acoustic window in the electromagnetic energy transmitter or transmitter array. In an additional embodiment, the electromagnetic energy transmitter or transmitter array is flexible to conform to a range of body surface shapes.

Other features and advantages of the invention will be apparent from the following detailed description, and from the claims.

### BRIEF DESCRIPTION OF THE DRAWINGS

FIG. 1 depicts the system and method for thermoacoustic signal generation and data acquisition for imaging and measurement. In FIG. 1: 101 indicates an ultrasound transducer used to receive thermoacoustic signals from the tissue; 102 indicates acoustic coupling liquid or gel between the transducer and the skin or tissue surface; 103 indicates an acoustic data acquisition system; 104 indicates a signal processor; 105 indicates a display apparatus; 106 indicates blood vessels in a tissue; 107 indicates a region of tissue containing blood vessels; 108 indicates an electromagnetic (EM) energy applicator, transducer, or antenna; 109 indicates a transmitter or power source for the EM applicator; and 110 indicates an injector to inject the contrast agent into the blood vessels.

FIG. 2 depicts an embodiment of a conformal, flexible electromagnetic source applicator. In FIG. 2: 21 indicates an electromagnetic (EM) applicator; 22 indicates an EM power source or transmitter; 23 indicates an ultrasound signal detection system; 24 indicates an ultrasound receiving transducer; 25 indicates an optional aperture or acoustic window in applicator (transparent to ultrasound); and 26 indicates the body being examined.

FIG. 3 is a graph of the intrinsic absorption properties of different types of tissue as a function of electromagnetic frequency.

FIG. 4 is a graph of the absorption properties of different tissues and contrast agents as a function of electromagnetic frequency.

FIG. 5 shows thermoacoustic data following pulsed radiofrequency irradiation of: a 2-mm tube containing physiological 0.9% saline surrounded by de-ionized water (upper left panel), a 2-mm tube containing 2% saline surrounded by physiological 0.9% saline (upper right panel); a 2-mm tube and a 3-mm containing de-ionized water surrounded by physiological 0.9% saline (lower left panel); and 2-mm tube containing light mineral oil surrounded by water (lower right panel).

FIG. 6 is an image of thermoacoustic data following pulsed radiofrequency irradiation of four 0.3-mm tubes containing 5× physiological saline (5% NaCl) surrounded by physiological 0.9% saline.

### DETAILED DESCRIPTION

Perfusion of blood in and through tissue is related to the health of that tissue. Perfusion, being a general term, is more specifically characterized by parameters that include BF (blood flow), BV (blood volume), MTT (mean transit time), and PA (permeability-surface area product). Variants and derived quantities (for example, dispersion of mean transit time) from these parameters also characterize perfusion tissue. As is known in the art, these measured parameters characterize tissue and can be used as a diagnostic to differentiate tissue types, e.g., healthy from diseased tissue, or necrotic from viable tissue.

The invention provides thermoacoustic methods for analyzing soft tissue and vasculature in a subject, imaging soft tissue or vasculature in a subject, determining blood flow in a tissue, and diagnosing a disease in a subject, and systems that perform these methods.

Thermoacoustic imaging, a general term encompassing photoacoustic, optoacoustic, and photothermoacoustic imaging, is a field of technology used in characterizing and imaging materials based on their electromagnetic absorption and thermal properties. To date, most other imaging modalities measure the same energy that was used as the input: optical systems input and receive light, ultrasound systems input and receive ultrasound; X-ray computed tomography systems input and receive X-rays; and magnetic resonance systems transmit and receive radiofrequency energy. Thermoacoustic imaging, as described herein, is a hybrid modality which transmits electromagnetic energy but receives acoustic energy.

The thermoacoustic technique transmits pulses of energy that are absorbed by the material of interest (e.g., any bodily tissue of interest in a patient). Typically, near-infrared, microwave, or radiofrequency electromagnetic waves are used, collectively referred to herein as electromagnetic (EM) energy. The absorbed energy causes immediate heating, thermal expansion, and generation of an acoustic pressure wave with temporal characteristics defined by the incident pulse. In one embodiment of this invention, a pulse with duration of less than one microsecond is used to produce broadband acoustic signals, including wavelengths of less than one millimeter, which can be processed to produce images with sub-millimeter spatial resolution. In other embodiments of the methods, the incident pulse of electromagnetic energy may be between 1 ns and 10 microseconds in duration. In another example, the electromagnetic energy may be administered in a pulse chain of multiple pulses, each of which may have the same or different pulse widths and the same or different interval between pulses.

Several configurations of the system are possible involving both fixed energy transmitting components or compact packaging enabling portability and point of care applications. In the fixed energy transmitting component configuration, the EM transmitting transducer is fixed, and the subject or tissue being imaged is placed in proximity of the transducer. In a point of care application, the transducer is integrated into a compact deformable enclosure and may be placed in direct contact of the subject in proximity of the tissue to be imaged.

### Methods for Analyzing a Soft Tissue and Vasculature

The invention provides methods for analyzing tissue or vasculature of a subject by: coupling an ultrasound transducer to the subject; delivering to the subject a contrast agent that changes the thermoacoustic signal generated by the soft tissue or vasculature; irradiating the soft tissue or vasculature with electromagnetic energy to generate the thermoacoustic signal; and detecting the thermoacoustic signal; thereby analyzing the soft tissue or vasculature using the thermoacoustic signal. Analysis may or may not include one-dimensional, two-dimensional, or three-dimensional image formation. Thermoacoustic imaging provides a spatial map of the relative energy absorption by tissue.

Non-invasive diagnostic imaging procedures are used widely in clinical practice to visualize and quantify: anatomy, physiology, tissue function, disease state, and response to therapy. A common requirement for many diagnostic and non-diagnostic imaging procedures is the ability to discriminate non-skeletal tissue types that have largely the same composition (protein, lipid, elastin, water, minerals, and collagen). The ability of a medical imaging system to discriminate these non-skeletal tissues is commonly referred to as “soft tissue contrast.” In practice, soft tissues may be discriminated when the signal difference between soft tissue types is greater than the variance in signal (i.e., noise). Several medical imaging modalities are routinely used in image guided procedures, screening, the diagnosis of disease, and monitoring of therapy. Magnetic resonance imaging (MRI) is the modality that provides the greatest magnitude of soft tissue contrast with endogenous contrast only. Other imaging modalities such as X-ray, computed tomography (CT), nuclear imaging, PET, and ultrasound have relatively poor soft tissue contrast compared to MRI. The introduction of an exogenous material (contrast agent) may increase soft tissue contrast. The differential vasculature (vessel density), vascular permeability, and tissue perfusion of soft tissues can be leveraged through the use of vascularly-administered exogenous agents. These exogenous agents may be administered through infusion or bolus injection, and are commonly used in clinical practice with MRI, X-ray, CT, nuclear imaging, PET, and ultrasound. Small molecule contrast agents may diffuse into interstitial spaces (extra-vascular contrast agents), while large molecule contrast agents remain in the vasculature (blood pool contrast agents) until they are broken down and/or excreted.

In the radio and microwave frequencies, endogenous energy absorption by tissue is dominated by ion concentration and dielectric absorption. The soft tissue contrast of a thermoacoustic imaging system may be increased by the introduction of an exogenous contrast agent that either increases or decreases the endogenous absorption rate of irradiating energy by a tissue or vasculature. The endogenous soft tissue contrast may be increased by the introduction of an exogenous contrast agent that increases or decreases ion concentration of the tissue or vasculature. A contrast agent with an ion concentration that is hyperionic compared to tissue or vasculature will increase the absorption rate of radiofrequency (RF) radiation by tissue or vasculature containing the exogenous contrast agent, while a contrast agent with hypo-ionic concentration compared to tissue or vasculature will decrease the absorption rate of RF radiation by tissue or vasculature containing the contrast agent. Alternatively, the introduction of an exogenous contrast agent that has lower dielectric absorption than water will decrease the absorption rate of microwave energy by tissue or vasculature. Similarly, an agent with higher dielectric absorption than water will increase the absorption rate of microwave energy by tissue or vasculature.

For example, a suitable contrast agent in connection with the thermoacoustic methods provided herein is an agent that has an increased dielectric absorption compared to soft tissue or vasculature, e.g., blood (e.g., a dielectric absorption that is at least 1.5-fold, 2.0-fold, 3.0-fold, 4.0-fold, 5.0-fold, 6.0-fold, 7.0-fold, 8.0-fold, 9.0-fold, 10-fold, 15-fold, 20-fold, 25-fold, 30-fold, 35-fold, 40-fold, 45-fold, 50-fold, 60-fold, 70-fold, 80-fold, 90-fold, or 100-fold greater than the dielectric absorption of soft tissue or vasculature), or an agent that has an increased ionic conductivity compared to soft tissue or vasculature (e.g., ionic conductivity that is at least 1.5-fold, 2.0-fold, 3.0-fold, 4.0-fold, 5.0-fold, 6.0-fold, 7.0-fold, 8.0-fold, 9.0-fold, 10-fold, 15-fold, 20-fold, 25-fold, 30-fold, 35-fold, 40-fold, 45-fold, 50-fold, 60-fold, 70-fold, 80-fold, 90-fold, or 100-fold greater than the ionic conductivity of soft tissue or vasculature).

Additional suitable contrast agents that may be used in the thermoacoustic methods provided herein are agents that have a decreased dielectric absorption compared to soft tissue or vasculature, e.g., blood (e.g., a dielectric absorption that is at least 1.5-fold, 2.0-fold, 3.0-fold, 4.0-fold, 5.0-fold, 6.0-fold, 7.0-fold, 8.0-fold, 9.0-fold, 10-fold, 15-fold, 20-fold, 25-fold, 30-fold, 35-fold, 40-fold, 45-fold, 50-fold, 60-fold, or 70-fold less than the dielectric absorption of soft tissue or vasculature) or have a decreased ionic conductivity compared to soft tissue or vasculature, e.g., blood (e.g., an ionic conductivity that is at least 1.5-fold, 2.0-fold, 3.0-fold, 4.0-fold, 5.0-fold, 6.0-fold, 7.0-fold, 8.0-fold, 9.0-fold, 10-fold, 15-fold, 20-fold, 25-fold, 30-fold, 35-fold, 40-fold, 45-fold, 50-fold, 60-fold, 70-fold, 80-fold, 90-fold, or 100-fold less than the ionic conductivity of soft tissue or vasculature). The above examples are not intended to limit the scope of the mechanisms described. Other agents and loss mechanisms may be employed to increase or decrease electromagnetic energy absorption in soft tissue or vasculature. Furthermore, the method is equally applicable to mechanisms that modify the intrinsic absorption rate or thermoacoustic efficiency of soft tissue or vasculature, such as changing the temperature or ion concentration.

The analysis of the thermoacoustic signal measured in the above methods may be used to determine one or more blood flow parameters in a subject. As is generally known in the art, BF is the volume flow of blood through the vasculature, comprising the large vessels, arteries, arterioles, capillaries, venules, veins, and venous sinuses. BF is usually normalized to a convenient volume of tissue and usually carries the unit of mL/min/100 g. BV is the fraction of a tissue of interest occupied by the blood in the vasculature (comprising the large vessels, arteries, arterioles, capillaries, venules, veins, and venous sinuses). It typically is expressed in units of mL/g or as a percentage. MTT recognizes that blood flows through multiple paths in tissue, so there does not exist a unique transit time from inlet to outlet, but rather a distribution of transit times. This distribution is represented by an average or mean transit time, being the mean of the distribution of transit times. The Central Volume Principle relates the parameters according to the relationship BF=BV/MTT.

The method for estimating blood flow parameters, specifically BF, BV, MTT, and PA, uses an injection of a bolus of contrast agent into the vasculature, either on the venous or arterial sides. The duration of the injection causes a time-varying concentration of contrast agent, Ca(t), in the arterial side upstream of the region of interest on the body. The duration of the injection is typically short in comparison with the duration of the physiological events being measured, such as MTT. The curve describing Ca(t) becomes convolved with the dispersion of the contrast agent in its progression through the tissue and vasculature in the region of interest. A sequence of thermoacoustic images measures the concentration, Q(t), of the contrast agent in the tissue and vasculature over time. The arterial concentration of the contrast agent over time Ca(t) is also measured, and the blood flow parameters in the tissue of interest are computed by deconvolution of Q(t) and Ca(t) and analysis of the resulting concentration curve, as is known in the art.

The method provides for the detection of the contrast agent in tissue and computes the tissue blood flow parameters. As with any analysis of the invention, the measured and computed parameters can be presented as numerical results, can be displayed as parameter-vs.-time plots, or can be images showing the spatial distribution of the parameters, or can be shown as images evolving in time (commonly called cineloops in the art).

The blood flow parameters (e.g., BF, BV, MTT and PA) determined in a subject may be compared to the blood flow parameters (e.g., BF, BV, MTT and PA) measured in a healthy subject or a control tissue in the same subject. In various embodiments of the methods provided by the invention, the contrast agent may be delivered to the subject prior to the start of irradiation with electromagnetic energy. In other examples of these methods, the contrast agent may be delivered to the subject after the start of irradiation with electromagnetic energy or delivered to the subject at the same time as the start of irradiation with electromagnetic energy.

The above methods may also be used to classify the tissue according to its blood flow parameters and may also use differences among the sequence of images to produce an angiogram image showing the blood vessels.

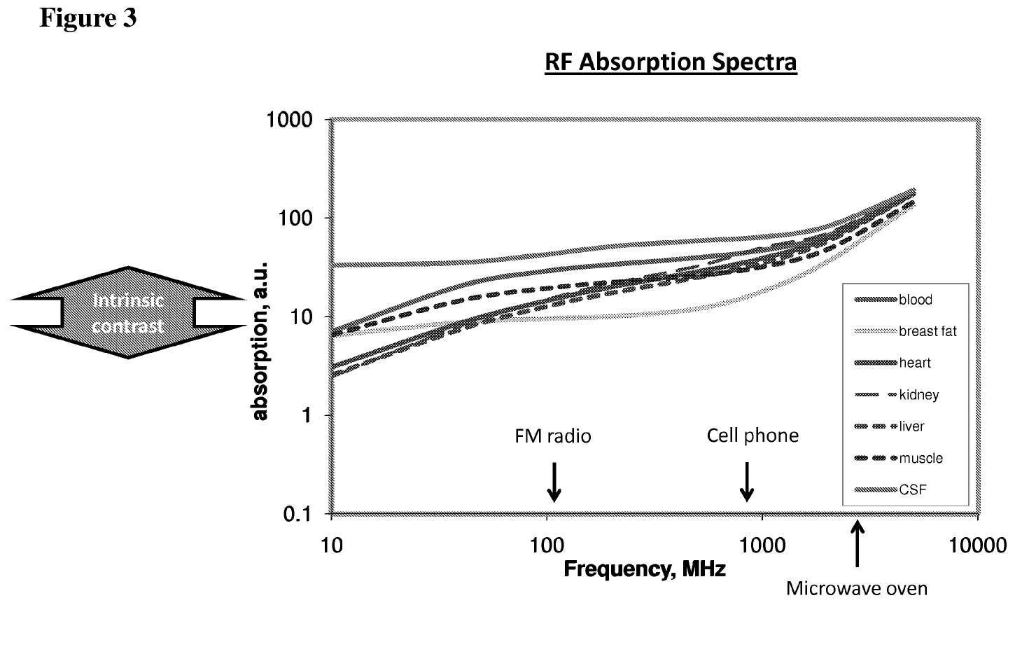

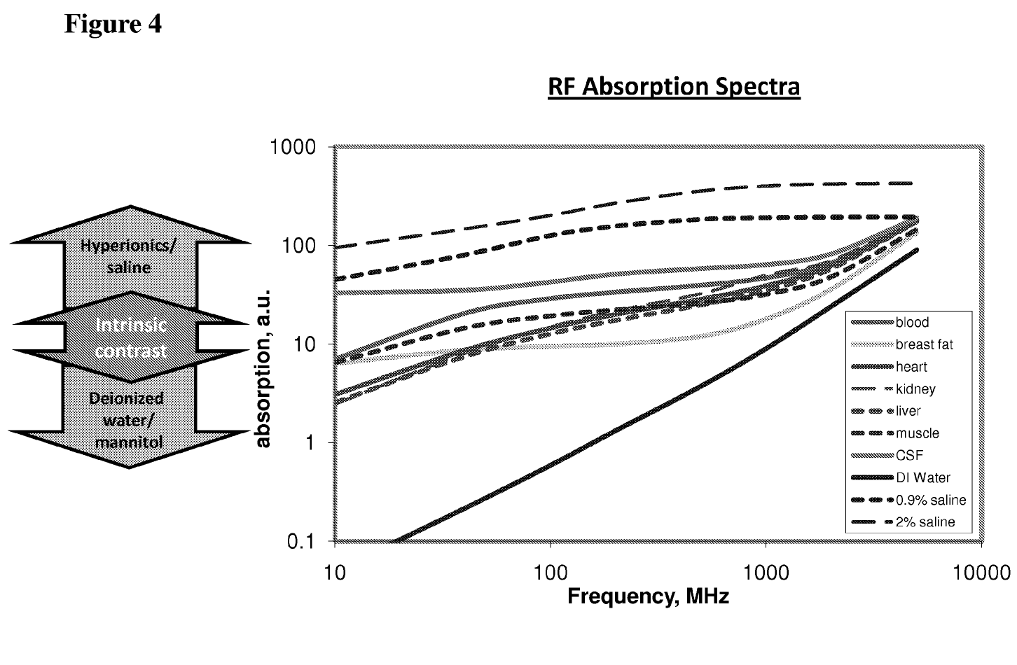

The contrast agent may be delivered as a bolus injection or via manual or mechanical infusion. Contrast agents that may be used with the provided methods include physiologically-tolerable contrast agents, that is, contrast agents that do not cause immediate or lasting deleterious effects to living organisms and that are generally regarded as safe. The principal property of a contrast agent is that it differs from blood and/or tissue in its absorption of the incident EM energy (e.g., radiofrequency energy waves). FIG. 3 depicts the absorption properties of several different tissues. Contrast agents differ in the absorption properties depending on the application and the wavelength of the EM energy (FIG. 4). Non-limiting examples of contrast agents that may be used in the methods include:
1) Physiologic saline solution, which may include sodium chloride solution, other salt solution, or which may be a composite of several salts or other materials, such as may be commonly available and accepted for use for other medical application, such as Ringer's or Hartmann's solutions.
2) Hyperionic solutions, which exhibit increased EM energy absorption compared to soft tissue or vasculature, e.g. blood. Hyperionic solutions may contain one or more salts including, for example, calcium chloride, calcium sulfate, calcium iodate, magnesium chloride, magnesium sulfate, copper sulfate, cuprous iodide, magnesium chloride, magnesium sulfate, magnesium phosphate, magnesium sulfate, manganese chloride, potassium chloride, potassium iodide, potassium iodate, potassium sulfate, and/or sodium phosphate.
3) Hypo-ionic solutions or non-ionic solutions, which exhibit decreased EM energy absorption to radiation compared to soft tissue or vasculature, e.g., blood, and serve as negative contrast agents (e.g., de-ionized water). Hypo-ionic solutions may contain the following salts in ionic concentrations less the physiological concentration: calcium chloride, calcium sulfate, calcium iodate, magnesium chloride, magnesium sulfate, copper sulfate, cuprous iodide, and magnesium chloride.
4) Low-conductivity isotonic solutions that do not promote substantial cell shrinkage (plasmolysis) or rupture (cytolysis) due to osmotic difference from soft tissue or vasculature, e.g., blood, but exhibit lower EM energy absorption than soft tissue or vasculature and may be used as negative contrast agents (e.g., solutions containing molecules that do not dissociate in water, such as solutions of 5% mannitol, 5% dextrose, 2.5% glycerol, or similar solutions).
5) Isotonic solutions, colloids, emulsions, suspensions, or mixtures that modify the EM energy absorption of soft tissue or vasculature and do not promote cell shrinkage (plasmolysis) or rupture (cytolysis) due to osmotic differences. Non-limiting examples of isotonic solutions include blood plasma substitutes (e.g., Voluven®, Haemaccel®, and Gelofusine®).
6) Suspensions or colloids of ferromagnetic and ferrimagnetic particles (e.g., uncoated magnetite, elemental iron, and magnetic iron oxide particles in starch, dextran, lipid, and polyacrylic).
7) Suspensions or colloids of non-magnetic particles with a dielectric loss different from soft tissue or vasculature, e.g., blood (e.g., enzyme-modified fats, maltoextran, malt extract, corn sugar, corn syrup, safflower oil, glycerol, and other lipids and oils).
8) Blood substitutes that exhibit a thermoacoustic response to EM energy different from soft tissue or vasculature, e.g., blood (e.g., perfluorocarbons, Hemopure®, Oxygent™, PolyHeme®, and Perftoran).
9) Dyes that absorb EM energies in the red or infrared region of the spectrum, such as Indocyanine Green and Evan's Blue. In certain embodiments of all of the methods, the use of dyes that absorb in the red or infrared region of the spectrum are excluded.
10) Agents that exhibit a thermoacoustic response different from soft tissue or vasculature, e.g., blood, by having a different thermal expansion coefficient, speed of sound, heat capacity, or, in general, a different Gruneisen coefficient.

In all cases, the contrast agent is different from soft tissue or vasculature, e.g., blood, in the sense that it has a different thermoacoustic response to the applied EM energy (e.g., radiofrequency wave energy), so it can be distinguished from soft tissue or vasculature (e.g., blood) by the difference in thermoacoustic signal produced. In some cases, the contrast agent moves out of the vasculature and into the interstitial space, thereby changing the endogenous absorption of the incident EM radiation. The physical mechanism that affords a difference in thermoacoustic response can be one or a combination of: a difference (an increase or decrease) in charge carrier density, such as ion density; a difference in dielectric absorption (loss tangent) (an increase or decrease); a difference in the speed of sound (increase or decrease); a difference in thermal expansion coefficient (an increase or decrease); a difference in heat capacity (an increase or decrease); or a difference in the molecular absorption (e.g., an optical or infrared dye). In non-limiting examples of the methods, a contrast agent is used that has increased absorbance relative to soft tissue or vasculature, e.g., blood (positive contrast agent) or decreased absorbance relative to soft tissue or vasculature, e.g., blood (negative contrast agent). Non-limiting examples of positive contrast agents include deionized water, an isotonic saline solution, safflower oil, or hypertonic saline. Non-limiting examples of negative contrast agents include hypotonic saline or an aqueous solution containing mannitol, dextrose, or glycerol.

As described in detail below, the electromagnetic energy in the above methods may be selected from near infrared light between 600 nm and 1000 nm, microwave energy between 1 GHz and 10 GHz, and radiowaves between 26 MHz and 1000 MHz.

The provided methods may be used to detect blood flow parameters in any tissue. Non-limiting examples of tissues that may be analyzed (irradiated) in the provided methods include heart, kidney, lung, esophagus, thymus, breast, prostate, brain, muscle, connective tissue, nervous tissue, epithelial tissue, bladder, gallbladder, intestine, liver, pancreas, spleen, stomach, testes, ovaries, and uterus.

Additional embodiments of the method further require generating a two- or three-dimensional image of the soft tissue or vasculature from the resulting thermoacoustic signal. For example, two or more images of the soft tissue or vasculature may be generated over time. In different embodiments of these methods, the two or more images may be collected over a uniform time interval (e.g., one image every second) or may be collected over a non-uniform time interval (e.g., in a first period, one or more images are obtained every second and, in a second time period, one or more images are obtained every two seconds).

### Methods for Diagnosing Disease

The invention also provides methods that indicate or aid in the diagnosis of a disease in a subject by: coupling an ultrasound transducer to the subject; delivering to the subject a contrast agent that changes a thermoacoustic signal generated by a tissue or vasculature; irradiating the tissue or vasculature with electromagnetic energy to generate the thermoacoustic signal; and detecting the thermoacoustic signal, thereby analyzing the soft tissue or vasculature using the thermoacoustic signal. All the above variations in the methods for analyzing a soft tissue or vasculature may be applied to the methods for indicating or diagnosing a disease in a subject.

Non-limiting examples of diseases that may be indicated or diagnosed using the methods of the invention include: cardiovascular disease, kidney disease, liver disease, stroke, and cancer. Non-limiting examples of cancer that may be detected by the provided methods include hepatocellular carcinomas, metastases, intrahepatic cholangiocarcinomas, liver hemangiomas, nonhemangiomatous benign lesions, adrenocortical carcinoma, anal cancer, appendix cancer, astrocytoma, atypical teratoid/rhabdoid tumor, basal cell carcinoma, bile duct cancer, bladder cancer, bone cancer, brain stem glioma, brain tumor, breast cancer, bronchial tumor, Burkitt lymphoma, carcinoid tumor, cervical cancer, chordoma, chronic lymphocytic leukemia, chronic myeloproliferative disorder, colon cancer, colorectal cancer, craniopharyngioma, cutaneous T-cell lymphoma, endometrial cancer, ependymoblastoma, ependymoma, esophageal cancer, Ewing sarcoma, extracranial germ cell tumor, extragonadal germ cell tumor, extrahepatic bile duct cancer, eye cancer, gallbladder cancer, gastric cancer, gastrointestinal cancer, germ cell tumor, gestational trophoblastic tumor, glioma, hairy cell leukemia, head and neck cancer, hepatocellular cancer, histiocytosis, Hodgkin lymphoma, hypopharyngeal cancer, intraocular melanoma, islet cell tumor, Kaposi sarcoma, kidney cancer, Langerhans cell histiocytosis, laryngeal cancer, acute lymphoblatic leukemia, chronic lymphocytic leukemia, lip and oral cavity cancer, liver cancer, lung cancer, non-Hodgkin lymphoma, macroglobulinemia, osteosarcoma, medulloblastoma, melanoma, merkel cell carcinoma, mesothelioma, mouth cancer, mycosis fungiodes, myelodysplastic syndrome, multiple myeloma, nasal cavity and paranasal sinus cancer, nasopharyngeal cancer, non-small cell lung cancer, oral cancer, oropharyngeal cancer, osteosarcoma, ovarian cancer, ovarian epithelial cancer, pancreatic cancer, papillomatosis, parathyroid cancer, penile cancer, pharyngeal cancer, pituitary tumor, prostate cancer, rectal cancer, renal cell cancer, retinoblastoma, rhabdomycosarcoma, salivary gland cancer, sarcoma, skin cancer, small intestine cancer, soft tissue sarcoma, testicular cancer, throat cancer, thomoma, thymic carcinoma, thyroid cancer, urethral cancer, uterine cancer, vaginal cancer, and Wilms tumor.

The thermoacoustic data (or blood flow parameters determined from the thermoacoustic data) derived from the patient tissue or vasculature may be compared to similar thermoacoustic data (or blood flow parameters determined from the thermoacoustic data) from a control sample, such as a subject not diagnosed with a disease, a control tissue from another part of the subject's body, or a prior set of thermoacoustic data collected from the same tissue in the subject on a prior date.

### Systems for Analyzing a Soft Tissue or Vasculature

The invention further provides systems for analyzing soft tissue or vasculature in a subject. These systems contain: an injector for delivering a contrast agent to the subject; an ultrasound receiving transducer or transducer array; an electromagnetic energy transmitter or transmitter array, wherein the electromagnetic energy transmitter or transmitter array administers pulsed electromagnetic energy to excite a thermoacoustic effect in the soft tissue or vasculature; and hardware or computer software to process a thermoacoustic signal generated by the soft tissue or vasculature.

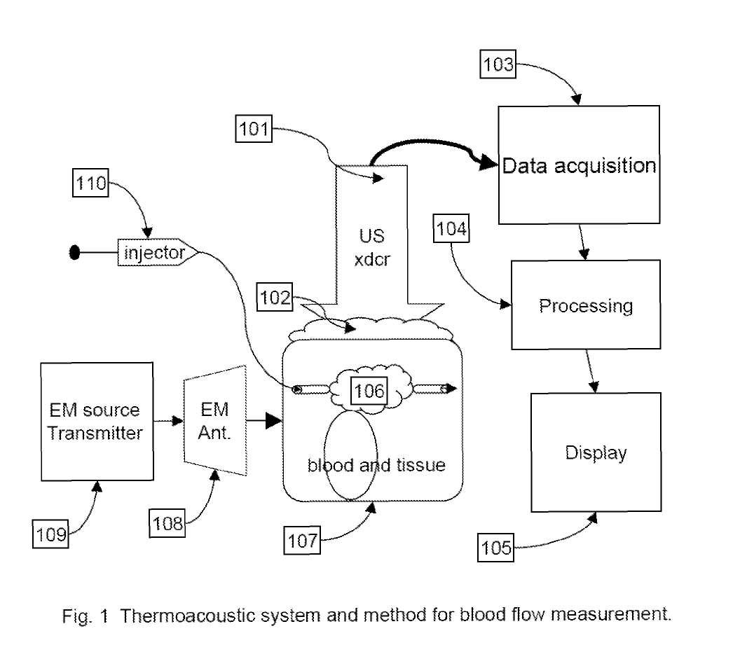

One non-limiting embodiment of a system provided by the invention is depicted in FIG. 1. Referring to FIG. 1, the system is used to measure blood flow parameters in a vascular region 106 in a body of tissue 107. An ultrasound receiving transducer 101 is coupled to the body with an acoustic coupling liguid or gel 102, which may be a commercially-available preparation or a specially-prepared formulation, or ordinary water. An electromagnetic transmitting transducer 108 is in close proximity to the body 107, to ensure good coupling of energy to the body. The EM transmitter or power source 109 supplies energy at the appropriate power, frequency, and pulse shape to the applicator, transducer, or antenna 108. During operation of the system, the source 109 and EM applicator 108 transmit pulses of EM energy into the body 107 containing blood and tissue of interest 106, stimulating thermoacoustic signals that are detected by the ultrasound transducer 101, then acquired and digitized by the data acquisition system 103. The signals are processed by the processor 104, and the results prepared and displayed 105. The injection of a contrast agent into the vasculature employs an injector 110, which may be motorized and automatic or may be manually driven by an operator.

The EM energy source is chosen 1) to provide a penetration depth in tissue suitable for a specific application, 2) to permit generation of individual pulses with a rise time short enough to produce acoustic pulses with detectable energy above 1 MHz, and 3) to allow absorption to provide contrast. At least three specific regions of the EM spectrum are useful for this purpose: 1) near infrared light between 600 nm and 1000 nm, which has a useful penetration depth up to 2 cm; 2) microwave energy between 1 GHz and 10 GHz, which exhibits good tissue contrast and penetration depth up to several centimeters; and 3) very high frequency and ultrahigh frequency radio waves between 26 MHz and 1000 MHz, which have frequencies high enough to produce the required short pulse rise time, and penetration depth of greater than several cm.

In one embodiment, the EM source is in the form of an array of antennas. The array is driven in phase and amplitude to minimize to a practical extent the electromagnetic field present at the location of the ultrasound transducer, in order to reduce the excitation of the detector element(s) and consequent transmission of an acoustic wave from the detector. The minimization of the EM field at the ultrasound transducer is also assisted by reducing the induced signal entering the receiver electronics during the EM pulse transmission, thus reducing or preventing risk of receiver damage or saturation and loss of sensitivity. An example is an array of a fixed geometry of discrete loop antennas; other examples include dipole, patch, microwave stripline, and transmission line antennas.

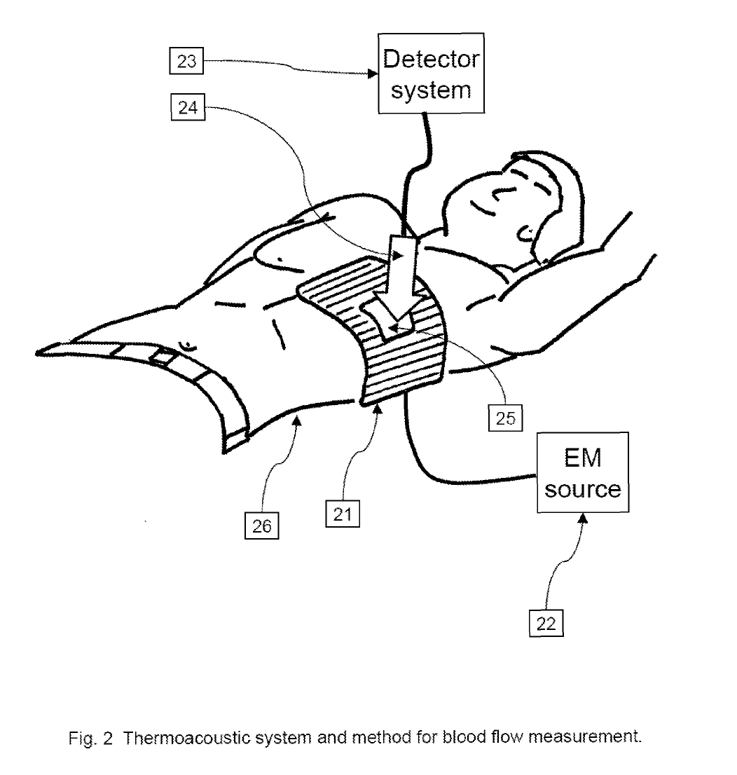

In one embodiment using radiofrequency, microwave, or optical energy, the EM source is in the form of an applicator of a conformal, optionally flexible, antenna or array of antennas or optical sources that can be applied to the surface of a body. The conformal array optionally may be provided with an aperture or acoustic window, through which the ultrasound detector can receive the thermoacoustic signals. One non-limiting example of such a system is shown in FIG. 2. Referring to FIG. 2, the applicator 21 is conforming to the body 26. The applicator may be pre-formed for a specific body part or size of body part, or it may be flexible to conform to a range of body surface shapes. The applicator 21 is driven by the EM source driver 22, as described herein. Also as described herein, the ultrasound transducer 24 receives thermoacoustic signals, which are then acquired and processed by the detector system 23. The thermoacoustic signals may be detected anywhere on the surface of the body by transducer 24. In one embodiment of an apparatus, the location for the transducer 24 is at an acoustic window 25 provided in the applicator 21, which may be located over the region of interest in the body. The acoustic window 25 may be simply an opening in the applicator 21 or it may be an acoustically-transparent membrane. In one embodiment, the applicator 21 is advantageously designed to minimize the power density or the field strength of its emissions in the location of the window 25, to reduce interference with the transducer 24.

The following system was employed for acquisition of in vitro data: 1) a pulsed radiofrequency source operating at 434 MHz; 2) a pair of opposing horn antennas tuned for 434 MHz and approximately 10 cm apart; 3) a rotating sample holder and 25-mm diameter target sample between the horn antennas; 4) a 5 MHz, 128-element 38-mm long ultrasonic receiver linear array, placed 30 mm from the center of rotation of the sample; 5) a 128-channel data acquisition system, digitizing at the rate of 20 MHz; and 6) a computer-based control system. In operation, the source provides pulses of electromagnetic energy approximately one microsecond in duration, with a risetime of less than 100 nanoseconds, at a rate up to 10 kHz, and a peak power up to 25 kilowatts. The ultrasonic receiver and data acquisition system records the acoustic signals produced as the electromagetic source is pulsed, and the sample is rotated within the electromagnetic field between the horn antennas. The recorded data may be processed to form cross-sectional images of the target sample. This system may be modified for in vivo use, e.g., by omitting the sample holder and placing the components relative to a subject.

As indicated above, the provided systems may include hardware or computer software for generating a two- or three-dimensional image from the thermoacoustic signal (e.g., generates a series of two or more images over time). The time interval between the two or more images may be uniform (e.g., one image every second) or non-uniform (e.g., a first period where images are produced every second and a second period where images are produced every two seconds).

Another example of the system includes hardware or a computer containing software that synchronizes the delivery of the contrast agent by the injector and the acquisition of the thermoacoustic signal by the ultrasound receiving transducer or transducer array. In another example, the system further includes hardware or a computer containing software for determining one or more blood flow parameters including Blood Flow (BF), Blood Volume (BV), Mean Transmit Time (MTT), and Tissue Permability-Surface Area product (PS) from thermoacoustic signals received by the ultrasound receiving transducer or transducer array.

In additional examples of the system, the electromagnetic energy transmitter or transmitter array is pre-formed for a specific body part or is flexible to conform to a range of body surface shapes. In another example of the system, the ultrasound receiving transducer or transducer array is connected to an acoustic window in the electromagnetic energy transmitter or transmitter array.

In one embodiment using radiofrequency or microwave energy where magnetic contrast agents are used, the EM source is in the form designed to maximize the magnetic field within the volume of tissue to be scanned.

In another embodiment using radiofrequency energy, where a contrast agent with high absorption due to ionic conductivity is used, the EM source is in the form designed to maximize the electric field within the volume of tissue to be scanned.

In another embodiment using microwave energy, where a contrast agent with high dielectric absorption is used, the EM source is in the form designed to maximize the electric field within the volume of tissue to be scanned.

In another embodiment using radiofrequency or microwave energy, where a magnetic contrast agent is used (e.g., a contrast agent containing a ferromagnetic or ferrimagnetic molecule), the EM source is in the form designed to produce a circularly polarized electromagnetic field within the volume of tissue to be scanned, with the objective to increase the difference in absorption between the contrast agent and the tissue.

In a further embodiment using microwave energy, where a ferromagnetic contrast agent is used, a complementary static magnetic field is used and the microwave frequency and magnetic field strength are adjusted to yield high absorption by the contrast agent, by exploiting the ferromagnetic resonance in the contrast agent.

In additional embodiments using radiofrequency or microwave energy, the EM source is advantageously in the form of a resonator with high quality factor to more efficiently couple the EM energy to the target absorbers.

The following examples provided below are not meant to be limiting and are meant to demonstrate only certain embodiments of the invention.

### EXAMPLES

### Example 1

### In Vitro Experiments Demonstrating a Thermoacoustic Method

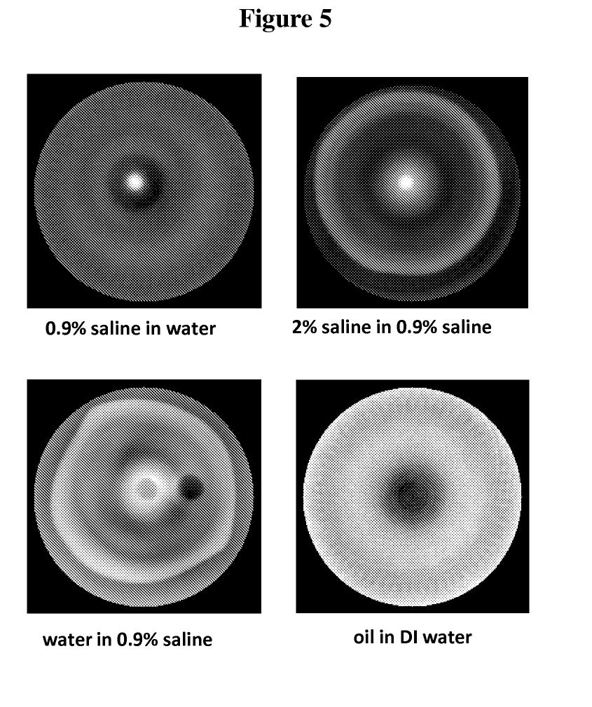

Experiments were performed in vitro to demonstrate the provided thermoacoustic method using a variety of contrast agents. In each experiment, a suitable contrast agent was placed in a 2-mm tube that was surrounded by a second aqueous solution and irradiated using pulsed radiofrequency energy, and resulting thermoacoustic data were gathered (FIG. 5). The data show positive (i.e., increased thermoacoustic signal) and negative (i.e., decreased thermoacoustic signal) depending on the contrast agent and surrounding medium used in each experiment. The upper left panel of FIG. 5 shows increased signal due to an increase in ion concentration and thus, conductivity and energy absorption, in a 2-mm tube of physiological 0.9% saline versus the surrounding de-ionized water. The upper right panel of FIG. 5 shows increased signal resulting from irradiation of a 2-mm tube containing 2% saline within an environment of physiological 0.9% saline. The lower left panel of FIG. 5 shows decreased signal resulting from the irradiation of a 2-mm tube containing de-ionized water compared to the surrounding environment of physiological 0.9% saline. The lower right panel of FIG. 5 shows decreased signal from the irradiation of a 2-mm tube containing light mineral oil compared to the surrounding environment of de-ionized water, due to the relative lack of dielectric absorption in the predominantly non-polar oil compared to the polar molecules in water.

The sum of these data show the ability of the thermoacoustic methods to detect the presence of these low toxicity contrast agents.

### Example 2

### Spatial Resolution of Data Provided by a Thermoacoustic Method

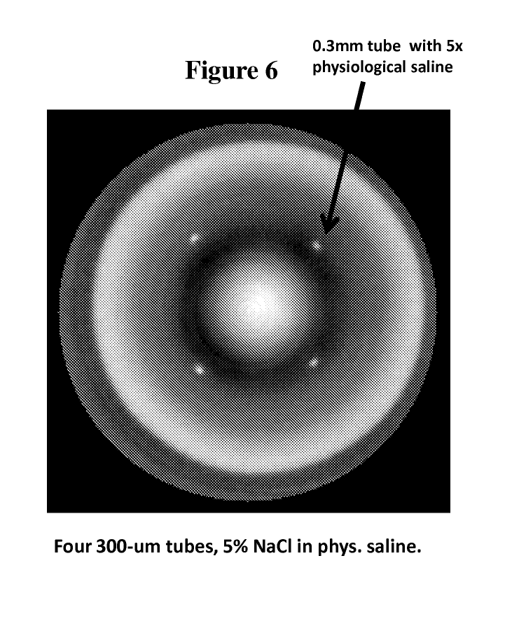

An in vitro experiment was performed to determine the spatial resolution of the data provided by thermoacoutic methods. In this experiment, four 0.3-mm tubes containing 5× physiological saline (5% NaCl) were placed in an environment of physiological saline, the tubes were irradiated with pulsed radiofrequency energy, and the resulting thermoacoustic data were collected (FIG. 6). The resulting data demonstrate that the thermoacoustic method is able to detect sub-millimeter structures at depth with very high contrast.

While the invention has been described in connection with specific embodiments thereof, it will be understood that it is capable of further modifications and this application is intended to cover any variations, uses, or adaptations of the invention following, in general, the principles of the invention and including such departures from the present disclosure that come within known or customary practice within the art to which the invention pertains and may be applied to the essential features hereinbefore set forth, and follows in the scope of the appended claims.

Other embodiments are within the appended claims.

1. A method for analyzing soft tissue or vasculature of a subject, comprising the steps of:
(a) coupling an ultrasound transducer to the subject;
(b) delivering to the subject a contrast agent that changes a thermoacoustic signal generated by the soft tissue or vasculature;
(c) irradiating the soft tissue or vasculature with electromagnetic energy to generate the thermoacoustic signal; and
(d) detecting the thermoacoustic signal, thereby analyzing the soft tissue or vasculature using the thermoacoustic signal.

2. The method of claim 1, wherein the electromagnetic energy is pulsed radiofrequency or microwave electromagnetic energy.

3. The method of claim 2, wherein the electromagnetic energy is pulsed radiofrequency energy.

4. The method of claim 2, wherein the electromagnetic energy is pulsed microwave energy.

5. The method of claim 1, wherein the delivery in step (b) is by bolus injection or by manual or mechanical infusion.

6. The method of claim 1, wherein said soft tissue is selected from the group consisting of: heart, kidney, lung, esophagus, thymus, breast, prostate, brain, muscle, nervous tissue, epithelial tissue, bladder, gallbladder, intestine, liver, pancreas, spleen, stomach, testes, ovaries, and uterus.

7. The method of claim 1, further comprising generating a two- or three-dimensional image of the soft tissue or vasculature from the thermoacoustic signal detected in step (d).

8. The method of claim 7, further comprising generating a series of two or more images over time.

9. The method of claim 8, wherein the time interval between said two or more images is uniform.

10. The method of claim 8, wherein the time interval between said two or more images is non-uniform.

11. The method of claim 1, further comprising determining one or more blood flow parameters from the vasculature.

12. The method of claim 11, wherein the one or more blood flow parameters are selected from the group consisting of Blood Flow (BF), Blood Volume (BV), Mean Transit Time (MTT), and Tissue Permeability-Surface Area product (PS).

13. The method of claim 11, wherein the determining of one or more blood flow parameters includes the step of generating a two- or three-dimensional image of the vasculature.

14. The method of claim 13, wherein said two- or three-dimensional image shows the location and size of the vasculature.

15. The method of claim 13, further comprising generating a series of two or more images over time.

16. The method of claim 15, wherein the time interval between said two or more images is uniform.

17. The method of claim 15, wherein the time interval between said two or more images is non-uniform.

18. The method of claim 1, wherein the analysis is indicative of a disease in the subject.

19. The method of claim 18, wherein said disease is cardiovascular disease, kidney disease, liver disease, stroke, or cancer.

20. The method of claim 19, wherein said cancer is selected from the group consisting of hepatocellular carcinomas, metastases, intrahepatic cholangiocarcinomas, liver hemangiomas, nonhemangiomatous benign lesions, adrenocortical carcinoma, anal cancer, appendix cancer, astrocytoma, atypical teratoid/rhabdoid tumor, basal cell carcinoma, bile duct cancer, bladder cancer, bone cancer, brain stem glioma, brain tumor, breast cancer, bronchial tumor, Burkitt lymphoma, carcinoid tumor, cervical cancer, chordoma, chronic lymphocytic leukemia, chronic myeloproliferative disorder, colon cancer, colorectal cancer, craniopharyngioma, cutaneous T-cell lymphoma, endometrial cancer, ependymoblastoma, ependymoma, esophageal cancer, Ewing sarcoma, extracranial germ cell tumor, extragonadal germ cell tumor, extrahepatic bile duct cancer, eye cancer, gallbladder cancer, gastric cancer, gastrointestinal cancer, germ cell tumor, gestational trophoblastic tumor, glioma, hairy cell leukemia, head and neck cancer, hepatocellular cancer, histiocytosis, Hodgkin lymphoma, hypopharyngeal cancer, intraocular melanoma, islet cell tumor, Kaposi sarcoma, kidney cancer, Langerhans cell histiocytosis, laryngeal cancer, acute lymphoblatic leukemia, chronic lymphocytic leukemia, lip and oral cavity cancer, liver cancer, lung cancer, non-Hodgkin lymphoma, macroglobulinemia, osteosarcoma, medulloblastoma, melanoma, merkel cell carcinoma, mesothelioma, mouth cancer, mycosis fungiodes, myelodysplastic syndrome, multiple myeloma, nasal cavity and paranasal sinus cancer, nasopharyngeal cancer, non-small cell lung cancer, oral cancer, oropharyngeal cancer, osteosarcoma, ovarian cancer, ovarian epithelial cancer, pancreatic cancer, papillomatosis, parathyroid cancer, penile cancer, pharyngeal cancer, pituitary tumor, prostate cancer, rectal cancer, renal cell cancer, retinoblastoma, rhabdomycosarcoma, salivary gland cancer, sarcoma, skin cancer, small intestine cancer, soft tissue sarcoma, testicular cancer, throat cancer, thomoma, thymic carcinoma, thyroid cancer, urethral cancer, uterine cancer, vaginal cancer, and Wilms tumor.

21. The method of claim 1, wherein said contrast agent has increased absorption of electromagnetic energy compared to the absorption of said soft tissue or vasculature.

22. The method of claim 21, wherein said electromagnetic energy is microwave or radiofrequency electromagnetic energy.

23. The method of claim 22, wherein said electromagnetic energy is radiofrequency energy.

24. The method of claim 22, wherein said electromagnetic energy is microwave energy.

25. The method of claim 1, wherein said contrast agent contains a ferromagnetic or ferrimagnetic particle.

26. The method of claim 22, wherein said contrast agent has an increased dielectric absorption or an increased ionic conductivity compared to said soft tissue or vasculature.

27. The method of claim 26, wherein said contrast agent is a hyperionic solution.

28. The method of claim 27, wherein said contrast agent is 5× physiological saline.

29. The method of claim 1, wherein said contrast agent has decreased absorption of electromagnetic energy compared to the absorption of said soft tissue or vasculature.

30. The method of claim 29, wherein said electromagnetic energy is microwave or radiofrequency electromagnetic energy.

31. The method of claim 30, wherein said electromagnetic energy is radiofrequency electromagnetic energy.

32. The method of claim 30, wherein said electromagnetic energy is microwave energy.

33. The method of claim 1, wherein said contrast agent has a decreased dielectric absorption or a decreased ionic conductivity compared to said soft tissue or vasculature.

34. The method of claim 33, wherein said contrast agent is a hypo-ionic solution.

35. The method of claim 33, wherein said contrast agent is an isotonic solution.

36. The method of claim 33, wherein said contrast agent is de-ionized osmolarity-balanced water, a solution containing safflower oil, or an aqueous solution containing mannitol, dextrose, or glycerol.

37. A system to analyze soft tissue or vasculature in a subject, comprising:
(i) an injector for delivering a contrast agent to said subject;
(ii) an ultrasound receiving transducer or transducer array;
(iii) an electromagnetic energy transmitter or transmitter array, wherein said electromagnetic energy transmitter or transmitter array administers pulsed electromagnetic energy to excite a thermoacoustic effect in the soft tissue or vasculature; and
(iv) hardware or a computer containing software to process a thermoacoustic signal generated by the soft tissue or vasculature.

38. The system of claim 37, wherein said electromagnetic energy is radiowave or radiofrequency energy.

39. The system of claim 38, wherein said electromagnetic energy is radiowave energy.

40. The system of claim 38, wherein said electromagnetic energy is radiofrequency energy.

41. The system of claim 37, further comprising hardware or computer containing software for generating a two- or three-dimensional image from the thermoacoustic signal.

42. The system of claim 41, wherein said hardware or computer generates a series of two or more images over time.

43. The system of claim 42, wherein the time interval between said two or more images is uniform.

44. The system of claim 42, wherein the time interval between said two or more images is non-uniform.

45. The system of claim 37, further comprising hardware or computer containing software that synchronizes the delivery of the contrast agent by the injector and the acquisition of the thermoacoustic signal by the ultrasound receiving transducer or transducer array.

46. The system of claim 37, further including hardware or a computer containing software for the determination of one or more blood flow parameters including Blood Flow (BF), Blood Volume (BV), Mean Transit Time (MTT), and Tissue Permeability-Surface Area product (PS) from thermoacoustic signals received by said ultrasound receiving transducer or transducer array.

47. The system of claim 37, wherein the electromagnetic energy transmitter or transmitter array is pre-formed for a specific body part.

48. The system of claim 37, wherein the ultrasound receiving transducer or transducer array is connected to an acoustic window in the electromagnetic energy transmitter or transmitter array.

49. The system of claim 37, wherein the electromagnetic energy transmitter or transmitter array is flexible to conform to a range of body surface shapes.
